(19) United States
(12) Patent Application Publication
(10) Pub. No.: US 2011/0112413 A1
(43) Pub. Date: May 12, 2011

(54) METHOD FOR AUTOMATED EKG ANALYSIS

(75) Inventors: Dorin Panescu, San Jose, CA (US); Bosko Bojovic, Belgrade (RS); Ljupco Hadzievski, Belgrade (RS); Uros Mitrovic, Belgrade (RS); Ihor Gussak, Morris TWP, NJ (US)

(73) Assignee: NEWCARDIO, INC, Santa Clara, CA (US)

(21) Appl. No.: 12/614,352

(22) Filed: Nov. 6, 2009

(51) Int. Cl. A61B 5/02

(52) U.S. Cl. 600/508

(57) ABSTRACT

Systems and methods for analyzing electronic cardiac signals for use in clinical diagnostics are described. System and method embodiments are configured to assist in the analysis of details of EKG signals and vector cardiograms to determine how patients should be categorized into specific cardiac risk categories, such as an acute coronary syndrome category. System configurations may comprise memory devices, computing systems, and EKG data sources positioned at various local or remote positions, and connected via various data connectivity modalities. Various parameters may be utilized to assist in the drawing of one or more conclusions regarding the cardiac condition of a patient.

### FIELD OF THE INVENTION

The invention relates generally to the analysis of electronic cardiac signals for use in clinical diagnostics, and specifically to systems and methods configured to assist in the analysis of details of EKG signals and vector cardiograms to determine how patients should be categorized into specific cardiac risk categories, such as an acute coronary syndrome category.

### BACKGROUND

Approximately 6.5 million patients present to U.S. emergency departments each year with chest pain. With the benefit of retrospective study, it is apparent that approximately 5.4 million of those patients do not have acute coronary symptoms (“ACS”), but rather some other clinical condition, such as heartburn, gall stones, or the like. Of the approximately 5.4 million, about 26% are ruled out by a first diagnostic triage in the emergency department, typically comprising at least a 12-lead electrocardiogram (or “EKG” or “ECG”) study. The remaining 74% of these 5.4 million patients are kept around in the hospitals for cardiac additional testing, until it is subsequently discovered, through additional time and testing, that most of these patients do not suffer from acute coronary syndrome. Given the present scenario of approximately 4,000 sophisticated emergency departments distributed throughout the United States, this is a load of approximately 1,000 patients per year, per emergency department, that are undergoing significant testing for acute coronary syndrome, only to find out later that most of such patients had no cardiac problems. There is a need for easily adoptable and better tools to minimally invasively, and efficiently, determine which patients are indeed suffering from genuine acute coronary syndrome. While some products, such as the special vest-type apparatus available under the tradename PrimeECG from Heartscape Technologies, Inc., and the multi-electrode panel system described in U.S. Pat. No. 6,584,343 have attempted to address some of the shortfallings of conventional EKG analysis, they require suboptimal changes in the EKG data acquisition process, such as a requirement of a specific vest type apparatus intercoupled to a specific data acquisition system. It would be preferable to require a minimal amount of change to such processes in clinical environments such as the emergency room.

### SUMMARY

One embodiment of the invention is directed to a method for detecting a first cardiac condition, comprising providing a quantity of 12-lead EKG data from a patient; constructing a 3-D representation of cardiac activity from the data; computing values for one or more parameters based upon at least one of said 3-D representation of cardiac activity and said EKG data; applying a multifactorial analysis protocol utilizing at least one of the values of the one or more parameters; and automatically drawing one or more conclusions regarding a first cardiac condition of the patient based at least in part upon the output of the multifactorial analysis protocol. The method may further comprise acquiring the quantity of 12-lead EKG data from the patient. Providing may comprise reconstructing a 12-lead EKG recording from a reduced cardiographic vector set from the patient. The method may further comprise receiving the vector set from another device. The other device may be selected from the group consisting of an implantable defibrillator, an implantable pacemaker, an implantable cardioverter, a portable EKG monitoring system, and a desktop EKG system. Providing may further comprise transmitting the EKG data from a plurality of electrodes to a computing system configured to construct the 3-D representation and compute values for the one or more parameters. Transmitting the EKG data from the electrodes to the computing system may occur in real or near-real time. Alternatively, transmitting the EKG data from the electrodes to the computing system may be delayed with utilization of a memory device interposed between the electrodes and computing system. Automatically drawing one or more conclusions regarding the first cardiac condition of the patient may further comprise categorizing the patient into a specific category of acute coronary syndrome relative to other patients. The electrodes and computing system may be interfaced with a connection selected from the group consisting of a local wired connection, a local wireless connection, a remote wired connection, and a remote wireless connection. The multifactorial analysis protocol may further utilize one or more parameters computed directly from the 12-lead EKG data. The method may further comprise providing feedback to a healthcare provider regarding the conclusions. The method may further comprise computing one or more confidence indices regarding the one or more conclusions based at least in part upon the one or more parameter values. The multifactorial analysis protocol may comprise quantitative analysis selected from the group consisting of discriminatory analysis, regression analysis, and hierarchical analysis. One of the one or more parameters may be a ratio-metric parameter based upon two or more aspects of the EKG data. One of the one or more parameters may be a ratio of Rmax relative to STshift or Tmax. One of the one or more parameters may be based upon the planarity of one or more vector cardiogram loops relative to a reference plane. One of the one or more parameters may be based upon the shape of one or more vector cardiogram loops relative to a reference shape, or relative to each other. The reference shape may comprise a circle or ellipse. One of the one or more parameters may be based upon the angular orientation of one or more vector cardiogram loops relative to a reference vector position, a reference plane or relative to each other. One of the one or more parameters may be based upon a Pardee sign analysis. One of the one or more parameters may be based upon a vector position relative to a computed center of a population distribution representative of a second cardiac condition, the vector selected from the group consisting of ST vector and T vector. The second cardiac condition may be selected from the group consisting of benign early repolarization, left ventricular hypertrophy, and right bundle branch block. The one of the one or more parameters of the multifactorial analysis protocol may be selected based upon a factor included in the group consisting of a patient's age, gender, race, residency, citizenship, occupation, and profession.

### BRIEF DESCRIPTION OF THE DRAWINGS

FIG. 1A illustrates a diagram of an EKG electrode configuration for a patient.

FIG. 1B illustrates a typical 12-lead EKG plot for a patient.

FIG. 2A illustrates various aspects and embodiments of a system for cardiac condition detection utilizing multifactorial analysis.

FIG. 2B illustrates various aspects and embodiments of a system for cardiac condition detection utilizing multifactorial analysis.

FIG. 2C illustrates various aspects and embodiments of a system for cardiac condition detection utilizing multifactorial analysis.

FIG. 3 illustrates a configuration for utilizing multifactorial analysis in cardiac condition detection with one or more EKG-based parameters.

FIG. 4 illustrates a 3-D representation of cardiac activity plotted for an operator of a user interface.

FIG. 5 illustrates various aspects of a parameter-based multifactorial analysis protocol.

FIGS. 6A and 6B illustrate differences in plane to plane angle for 3-D representations of cardiac activity.

FIG. 7 illustrates a Pardee analysis parameter application.

FIG. 8 illustrates a Gamma 2D parameter determination technique.

FIG. 9 illustrates an exemplary multifactorial analysis protocol which utilizes a Gamma 2D parameter in discriminant analysis.

FIG. 10 illustrates an exemplary multifactorial analysis protocol which utilizes a hierarchical decision pathway.

FIG. 11 illustrates a use of regression analysis to test candidate parameters utilizing preexisting data.

### DETAILED DESCRIPTION

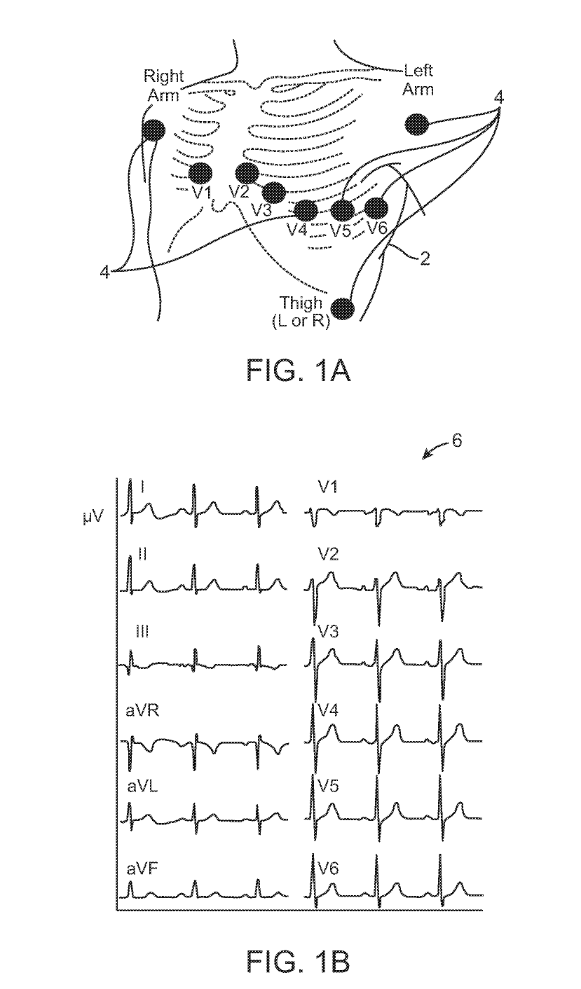

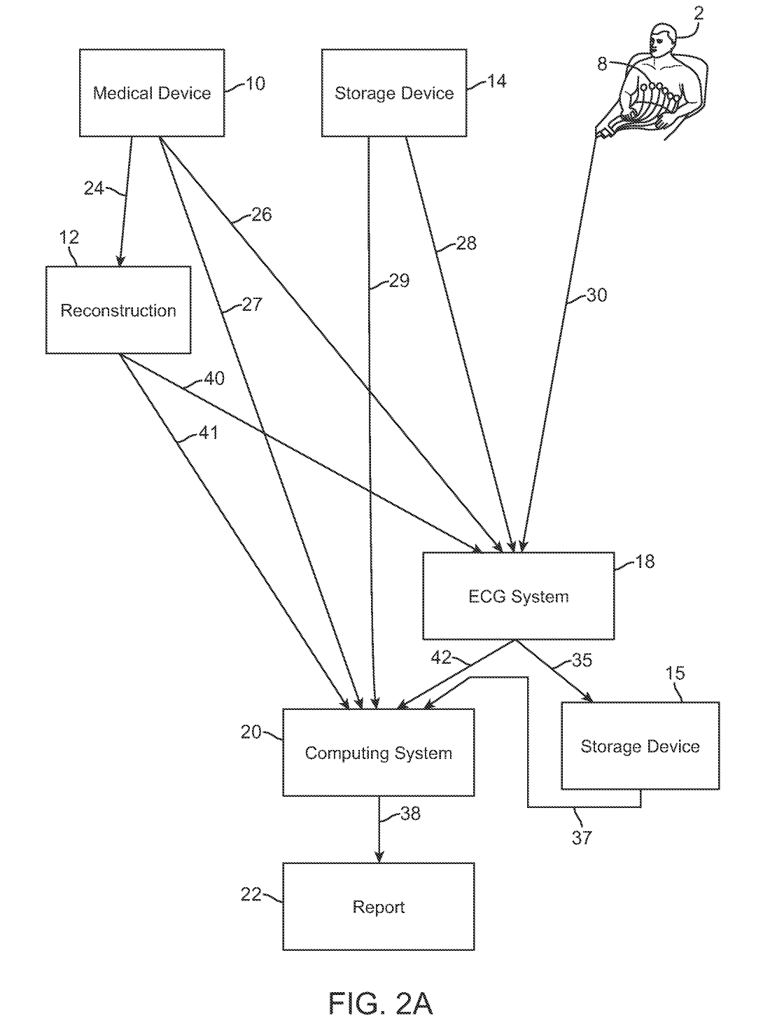

Referring to FIG. 1A, a typical EKG electrode location (4) configuration is depicted for capturing a standard 12-lead EKG from a patient (2). The data from the electrodes may be utilized with a conventional strip chart recorder or plotter to create an output (6) such as that depicted in FIG. 1B. As described above, aspects of this kind of conventional EKG output (6) are very useful in many types of diagnostics, and as it turns out, EKG data is rich with information beyond conventional EKG application, as described, for example, in U.S. patent application Ser. No. 12/484,156, entitled “Method for quantitative assessment of cardiac electrical events”, which is incorporated by reference herein in its entirety. To proceed with utilization of such data in further processing, an immediate challenge is capturing such data.

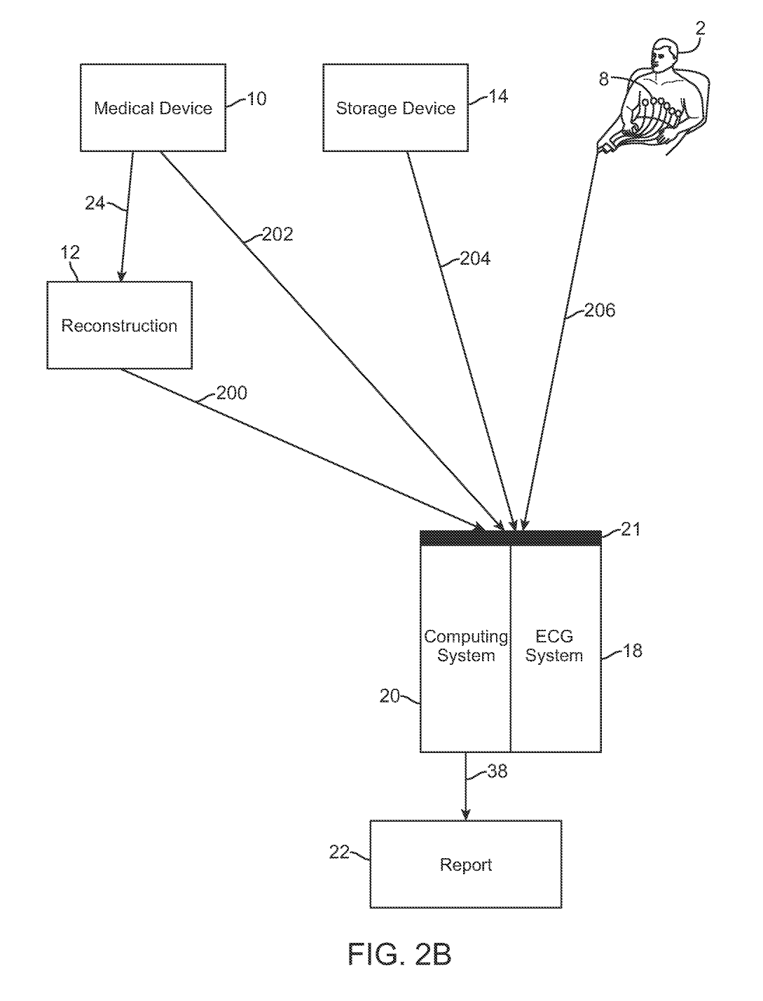

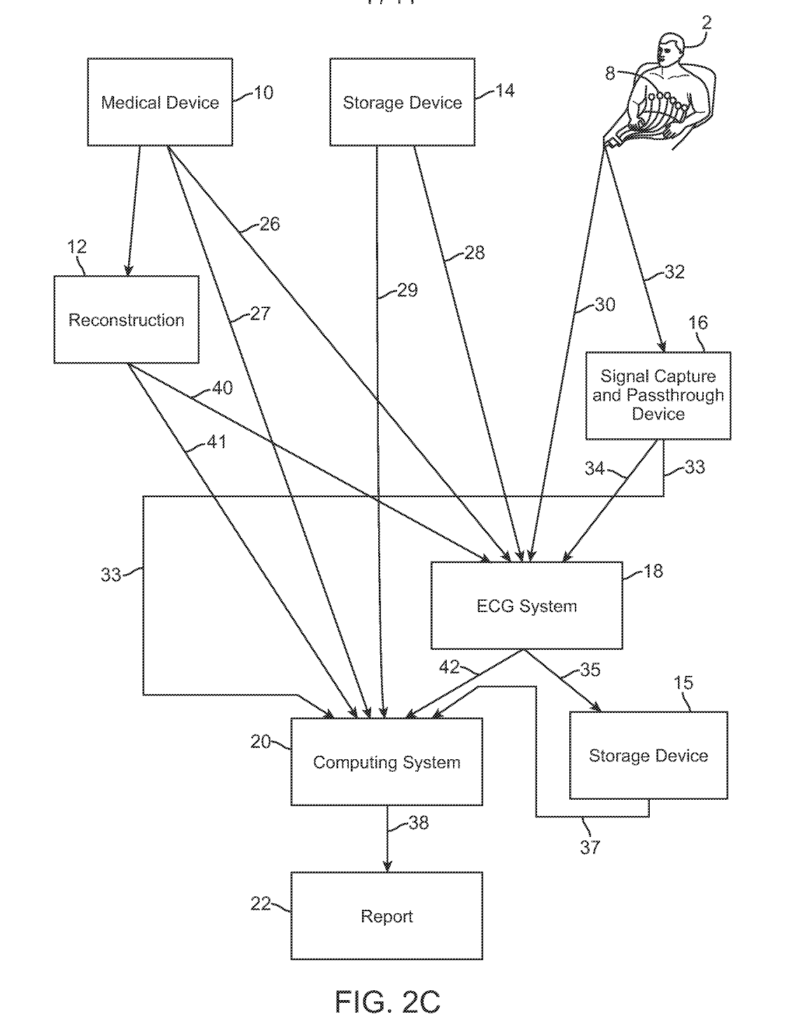

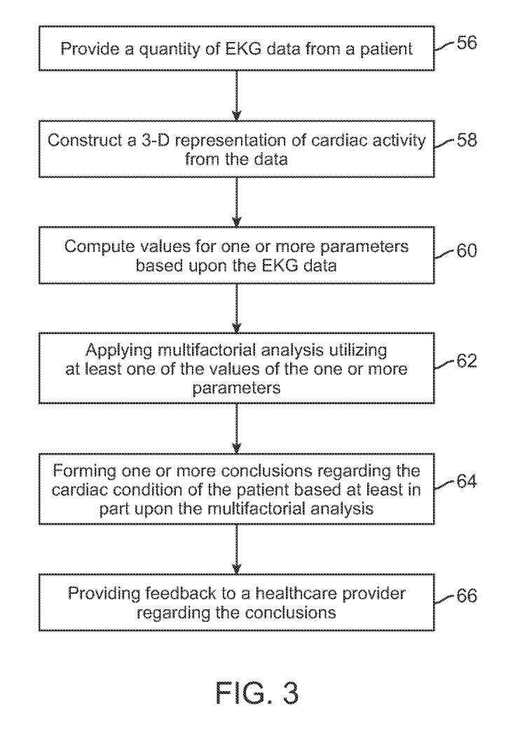

Referring to FIG. 2A, in one embodiment the data may be directed (30) from the patient (2) EKG electrodes (8) to an EKG system, such as those available from the Prucka Engineering division of GE Healthcare, Inc., and thereafter passed via a connection (42), in a form such as an electronic output file, to a computing system (20) configured to conduct detailed analysis of such data and ultimately facilitate production (38) of a report (22) configured to provide diagnostic information and/or conclusions to a healthcare operator. The EKG system (18) may be configured to filter, conduct an analog to digital conversion of, or store the pertinent EKG data before or after passing it to the computing system (20). The EKG system (18) may also be configured to pass (35) the data to a storage device (15) which may be utilized to provide the computing system (20) with access to such data through a connection (37) to the storage device (15), for example for a clinical scenario wherein a cardiologist wishes to review data and cases from an emergency department in an offline review scenario. Each of the connections between nodes, such as the patient, EKG system, computing system, storage device, and reporting mechanism, as well as other depicted devices, such as an additional storage device (14) and a medical device (10), may be conducted with a local wired connection, a local wireless connection, a remote wired connection, or a remote wireless connection, utilizing modern information technology infrastructure. In another embodiment, such connection may be manually conducted by virtue of a memory device configured to be used to transiently move data from one node to the next. Data may be moved between devices in many ways, such as realtime, near-real-time, in transient packets, by manual storage devices transiently.

In another embodiment, the source of data may not be a live patient (2), but rather a device capable of providing EKG-related data which may be dispatched to other devices and/or stored upon memory which may be coupled to or reside within such device. For example, referring to FIG. 2A, in one embodiment, the source may comprise a storage device (14) such as a flash memory or hard disk drive, capable of storing significant amounts of information, and preferably connected (28, 29) via one of the aforementioned connection types to an EKG system (18) or other networked device such as a computing system (20). In another embodiment, the source of data may comprise a medical device (10), such as a holter monitor, or a prosthesis, such as a defibrillator, pacemaker, or cardioverter which may or may not have a memory comprising stored EKG data, or stored reduced cardiographic vector set data, which may be utilized by a downstream computing device such as the EKG system (18) or computing system (20). Such device (10) may comprise a processor or microcontroller, and/or a memory device or interface. In one embodiment, the medical device (10) may comprise a product such as that available from NewCardio, Inc. under the tradename CardioBip®, described, for example, in U.S. patent application Ser. No. 10/568,868 by Bosko Bojovic, filed 2-21-06, incorporated by reference herein in its entirety; such device uses three non-standard EKG vectors and a reconstruction algorithm to produce a reconstructed 12-lead EKG recording from the three non-standard vectors. Preferably such device (10) is also connected (26, 27, 24) to other systems, such as the EKG system (18), computing system (20), or an intermediate computing device (12) configured for reconstructing a multi-lead EKG dataset, such as a 12-lead EKG dataset, from the reduced cardiographic vector set data which may be passed to it over a connection (40, 41). The intermediate computing device (12) may be incorporated or integrated into the medical device (10), the ECG system (18) or the computing system (20). Alternatively, its data may be stored in a storage device such as those illustrated as elements 14 and 15. Reconstruction of a multi-lead EKG dataset using reduced cardiographic vector set data from devices such as pacemakers or defibrillators has been discussed, for example, by Kachenoura et al in “Using Intracardiac Vectorcardiographic Loop for Surface ECG Synthesis”, EURASIP Journals on Advances in Signal Processing, Volume 2008, Article ID 410630, which is incorporated by reference herein in its entirety. Each of the connections referred to herein, such as those described above in reference to FIG. 2A (41, 40, 27, 26, 29, 28, 30, 42, 35, 38), may be configured as a local wired connection, a local wireless connection, a remote wired connection, or a remote wireless connection, utilizing modern information technology infrastructure.

Referring to FIG. 2B, another embodiment similar to that depicted in FIG. 2A is depicted, with the exception that the embodiment of FIG. 2B features a computing system (20) that is closely integrated with the EKG system (18). In one embodiment, the computing system (20) may comprise a module housed within the housing of the EKG system (18). In another embodiment, the computing system (20) and EKG system (18) are directly coupled or directly physically integrated relative to each other. In another embodiment, the computing system (20) may comprise a removable module operatively coupled to the EKG system (18). Referring to FIG. 2B, an input interface (21) is depicted for capturing incoming connections and data. The integrated systems may share an integrated input interface (21), as illustrated in FIG. 2B, or may each have their own input interface. With a tight integration of the computing system (20) and EKG system (18), data may be shared and moved between both systems. A unified input interface (21) facilitates simplified connections (206, 204, 202, 200) with sources such as patient electrodes (8), storage devices (14), medical devices (10), and other devices such as an intermediate reconstruction device (12). In another embodiment, the EKG system (18) has its own front end interface to directly accept signals from EKG electrodes (8) and the computing system (20) has its own digital interface (e.g. USB port, Bluetooth, wired, wireless, etc.) to directly accept information and/or signals from a storage device (14), from a medical device (10), and/or from a 12-lead EKG reconstruction device or subsystem (12). In such preferred embodiment the interface (21) would be split, rather than unified as depicted in FIG. 2B, to address the needs of systems (18) and (20). In one embodiment, systems (18) and (20) and the elements of interface (21) are all coupled or physically integrated in the same housing.

Referring to FIG. 2C, another embodiment is depicted illustrating that EKG related data from any of the depicted sources (8, 10, 12, 14, 15, 16) may be processed by the computing system (20) in parallel to connectivity to the EKG system (18) given suitable connections (37, 29, 27, 41, 33) for the computing system (20) and suitable connections (34, 30, 28, 26, 40) for the EKG system (18); the EKG system (18) and computing system (20) may also be operably coupled (42) to share information; in another embodiment they remain independent and the direct connection (42) is nonexistent.

Referring again to FIG. 2C, in another embodiment, signals may be passed (32) from the patient (2) electrodes (8) to an intermediate device (16) configured to store and/or transmit (33, 34) such data to the computing system (20) or EKG system (18). The intermediate device (16) may, for example, comprise a mini-EKG system that provides 12-lead EKG data to the computing system (20). At the same time, intermediate device (16) may pass through the signals from patient electrodes (8) to a standard EKG system (18). In such system configuration, data connection (42) may not be necessary. The intermediate device (16) may also have a low-power flash memory device along with a transmission bus, such as a wired or wireless transceiver bus, configured to interface with the EKG system (18), computing system (20), or other connections or devices to which the EKG data may be directed. In one embodiment, the intermediate device (16) may comprise analog front-end electronics, protection networks (e.g. against defibrillation shocks, electrostatic discharges, etc.), amplifiers, a microcontroller or microprocessor capable of various levels of processing of the data, such as analog to digital conversion and/or digital or analog filtering of various configurations, before dispatch to other connected systems.

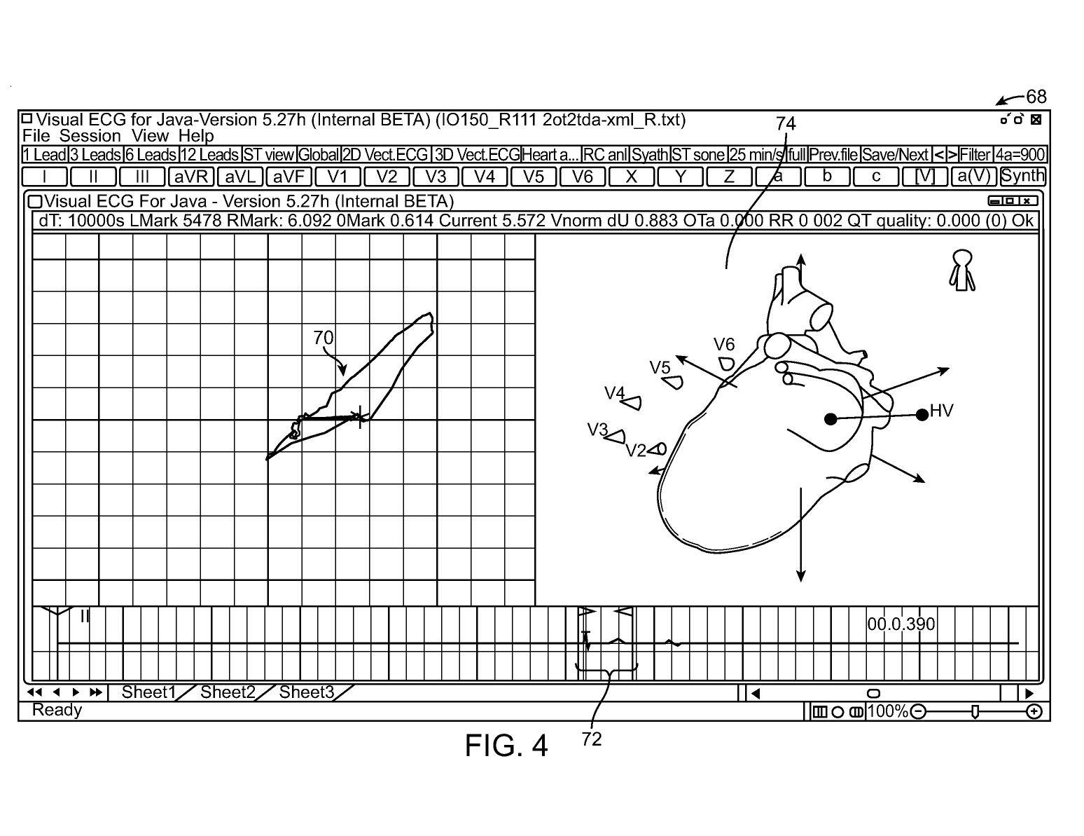

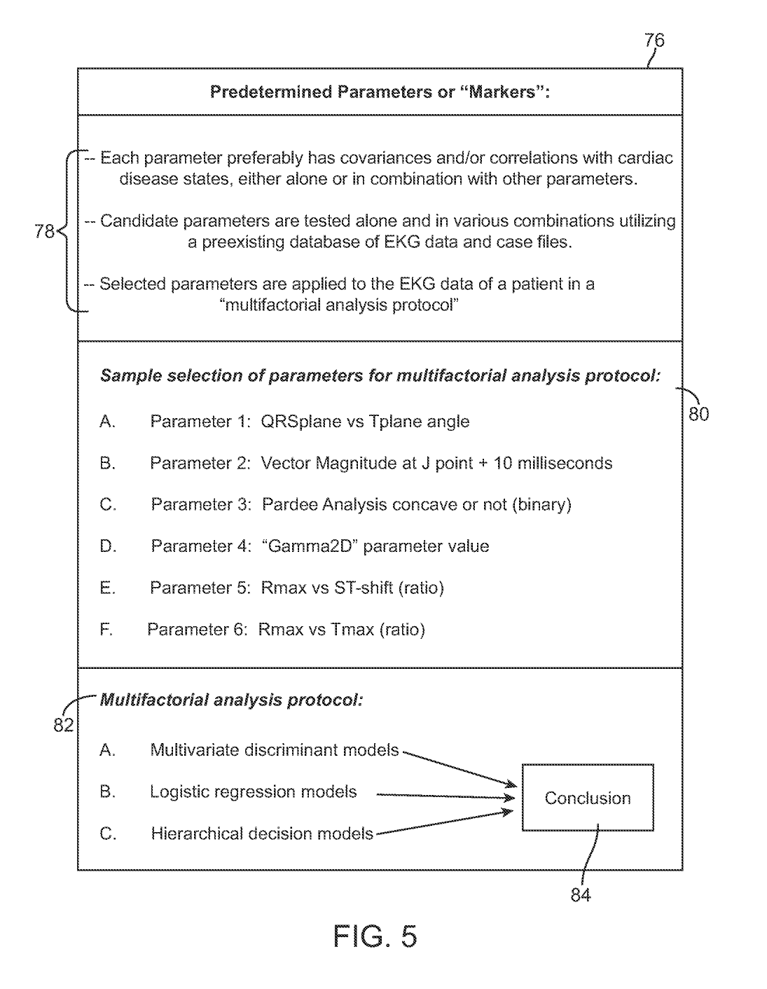

Referring to FIG. 3, systems such as those described in reference to FIGS. 2A-2C may be utilized to provide valuable feedback for healthcare providers (66). As shown in FIG. 3, a pertinent quantity of patient-related EKG data is provided (56). Utilizing this data, a three-dimensional (“3-D”) representation of cardiac activity may be constructed from the data (58). Subsequently, values for one or more preselected parameters may be computed utilizing the EKG data (60) (e.g. 3-D EKG data, 12-lead EKG data, or reconstructed 12-lead EKG). With such parameter values in hand, multifactorial analysis may be conducted (62) utilizing at least one of the values of the one or more parameters, and, in accordance with a particular multifactorial analysis protocol, one or more conclusions regarding the cardiac first condition of the patient may be drawn (64), based at least in part upon the multifactorial parameter-based analysis. The step of creating a 3-D representation of cardiac activity may be conducted utilizing 3-D vector cardiography computer software on a computing system, such as those described in reference to FIGS. 2A-2C, with software such as that available from NewCardio, Inc., the assignee, and described at least in part in the aforementioned incorporated patent application. A typical 3-D representation of cardiac activity utilizing such tools is depicted in FIG. 4, wherein the user interface (68) is configured to display a 3-D vector diagram (74), a plot (72) of a particular 12-lead trace portion being observed, and a loop diagram (70) pertinent to the portion. It is understood that such display is not required, or limiting, for the concept of 3-D representation of cardiac activity or for the operation of the invention. In one embodiment, referring to FIG. 3, the only visual output presented to the user (e.g. cardiologist, technician, emergency department doctor) may be in the form of a paper report coming out at step (66). A display, such as that illustrated in FIG. 4, may provide enhancing information, such as showing to the medical staff and estimated location of a cardiac infarct. The scope of this invention is not limited to visual or displayable types of 3-D representation of cardiac activity. Without limitation, computation of angles between QRS and T loops, for example, constitutes a 3-D representation of cardiac activity. Similarly, as in another example, computation of the magnitude of the cardiac vector constitutes a 3-D representation of cardiac activity. Yet as another example, conversion of a standard or reconstructed 12-lead EKG into X, Y, Z vectorcardiographic elements constitutes a 3-D representation of cardiac activity. Such transformation may be implemented, for example, as described by Dower in U.S. Pat. No. 4,850,370.

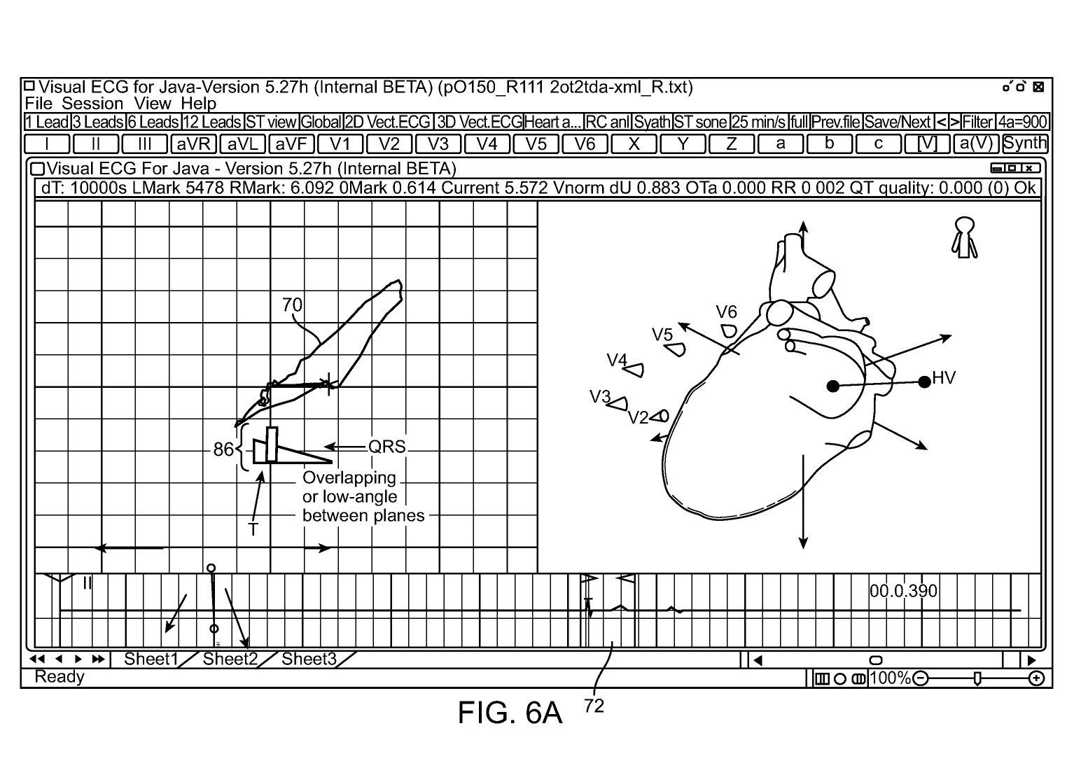

Referring to FIG. 5, the aforementioned predetermined parameters or “markers” preferably are selected for their ability to assist in the clinical diagnosis of patients in a particular group, versus patients not in such group. As shown in the table (76) of FIG. 5, each selected parameter preferably has several characteristics (78). Referring to FIG. 5, covariances and/or correlations with cardiac disease states, such as acute coronary syndrome, either alone, or in combination with other parameters are preferred; further, candidate parameters preferably are tested alone and in various combinations/permutations utilizing a preexisting database of EKG data and case files to determine which combinations and/or permutations have the best resolution in terms of the desired results. After such preferred combinations and/or permutations have been determined, they may be utilized by a computing system and applied to the EKG-related data of a particular patient in a multifactorial analysis protocol wherein more than one parameter-based sub-analysis is combined to create a decision analysis conclusion.

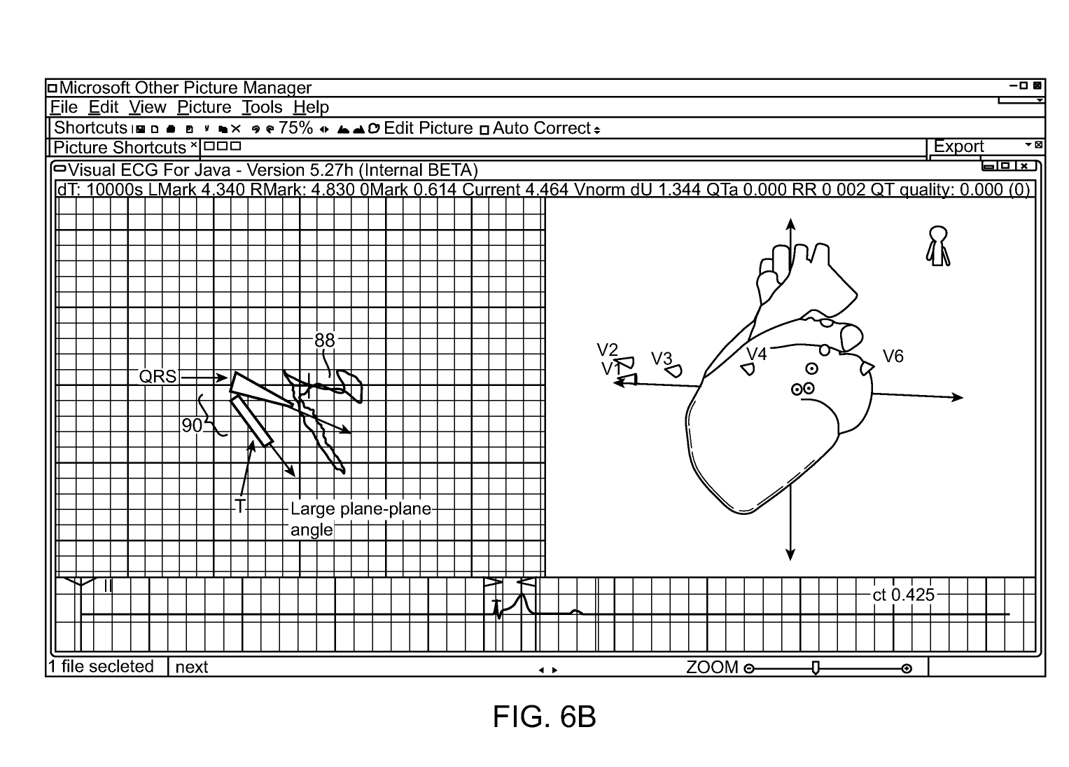

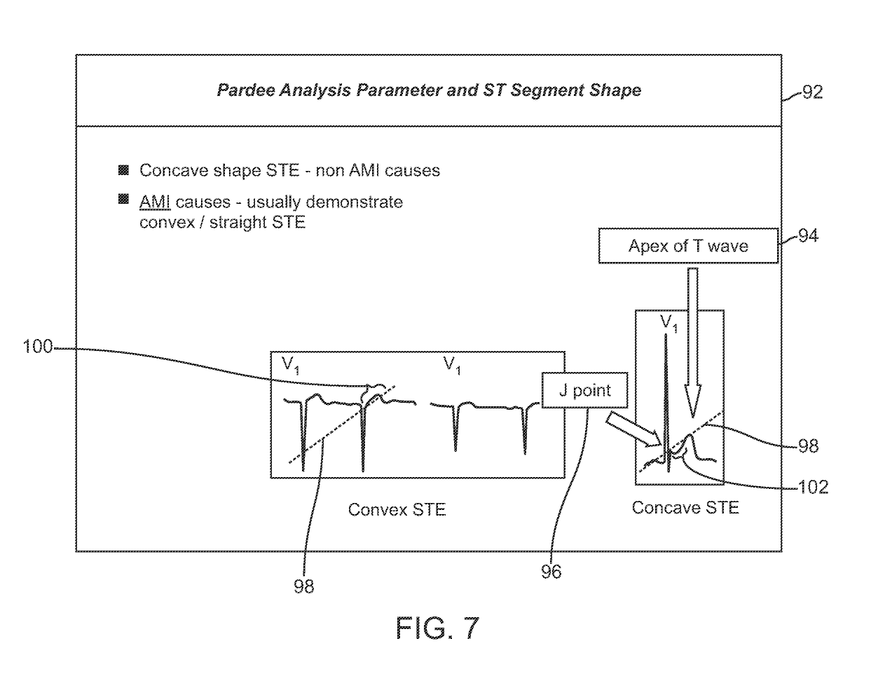

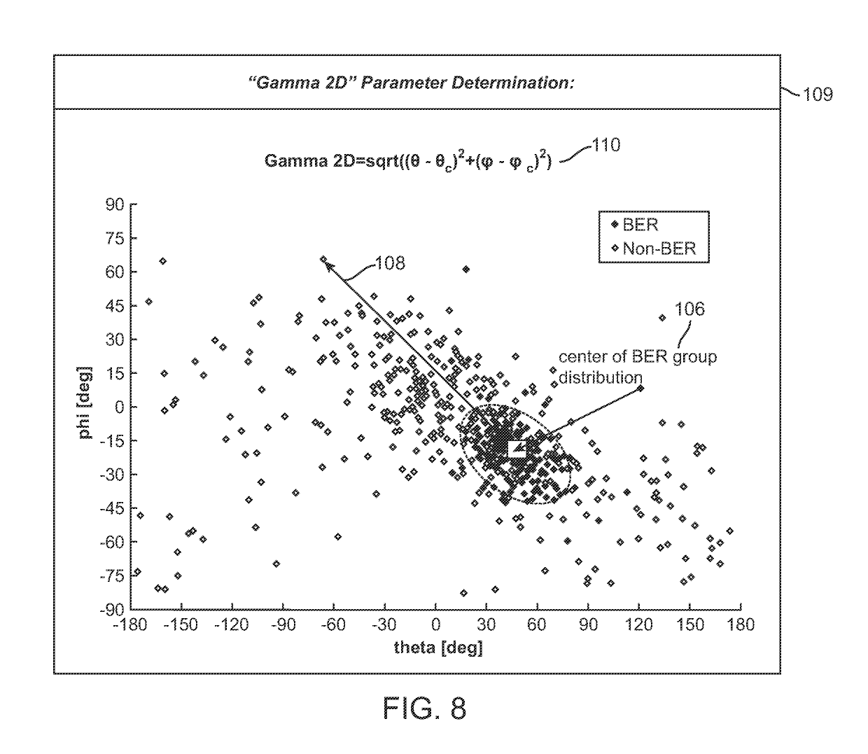

We have found in our experimentation that many candidate parameters or markers are useful in conducting cardiac EKG diagnostic analysis. For example, referring to FIG. 5, a listing (80) of a few is depicted, including a ratio of QRS plane angle versus Tplane angle, as described further in reference to FIGS. 6A and 6b, the QRS plane and Tplane angles being available from 3-D cardiography analysis; the vector magnitude from 3-D cardiography analysis at a point 10 milliseconds after the J-point on EKG; a determination (binary) of Pardee type concavity or not, from either the EKG data or the 3-D cardiography analysis, as described further in reference to FIG. 7; a “Gamma 2D” parameter value, as described further in reference to FIG. 8; and ratio-metric parameters such as the ratio of Rmax versus ST-shift from the EKG data, or the ratio of Rmax versus Tmax from the EKG data. The term Rmax refers to the peak of an R wave computed on the 3-D EKG representation (e.g., on the magnitude of the cardiac 3-D vector); the term ST-shift refers to the shift seen in the ST segment of the EKG vector magnitude; the term Tmax refers to the peak of the T wave of the EKG vector magnitude. Referring again to the table (76) in FIG. 5, a multifactorial analysis protocol (82) may comprise multivariate discriminant models, regression models, support vector machine models, and/or hierarchical decision models, to employ the various parameter values in furtherance of a clinically impactful conclusion (84). Further, in one embodiment, one or more confidence indices are computed regarding one or more of the conclusions based at least in part upon the one or more parameter values, preferably using further numerical analysis. For example, female patients younger than 65 years of age that present to emergency departments with non ST elevated myocardial infarction (NSTEMI) typically present confounding EKG morphologies. In one embodiment, applying multifactorial analysis to process data from such a patient, a myocardial infarction detection may be hypothetically rendered, and such conclusion may have a lower than average probability of being correct due to the confounding issues. One embodiment may be configured to utilize a self-computed confidence threshold that estimates the chances of its output being correct. If the chances of providing a correct detection output fall below this threshold, then the system may be configured to advise the healthcare provider of the detection result and of the decreased confidence level. In one embodiment, parameters in multifactorial analysis may be selected based upon a factor such as patient age, gender, race, residency, citizenship, occupation, or profession.

Referring to FIGS. 6A and 6B, loop plots may be utilized as parameters or elements thereof. For example, the angle between the QRS plane (or just the subplane corresponding to the QR portion) and T plane, different between the two specimens depicted in FIGS. 6A and 6B, may be utilized as a parameter. In non-ACS patients, it is expected that the QRS plane (or the QR subplane) and the T plane form a relatively low angle. This angle has been observed to increase in patients with ACS. In one embodiment, a threshold in the range of 20°-40° may be used to separate ACS from non-ACS patients. In addition to loop plane angulation, loop planarity (i.e., how planar is the loop), and loop shape, such as circularity or correspondence with an elliptical shape (i.e., how close is the loop to the shape of a circle or ellipse), may be utilized as parameters. For example, non-ACS patients tend to have QRS and T loops that are close to planar. Conversely, ACS patients tend to have QRS and T loops with geometric deviations from planar figures. To establish planarity, a summation of unsigned distances of points on the loop with respect to a reference plane, such as the principal component analysis plane, may be used as a planarity index. The lower the sum, the more planar the loop would be. As shown in FIG. 6A, the QRS loop (70) is approximately planar. The depicted loops were constructed from non-ACS EKG data, based on the process described in reference to FIG. 3. FIG. 6B illustrates a QRS loop (88) that cannot be reasonably approximated as planar. The loops in FIG. 6B were constructed from EKG data associated with an ACS patient, based also upon the process described in reference to FIG. 3. Also illustrated in FIGS. 6A and 6B, the QRS-T angle (86 and 90, respectively) has a relatively low value for the non-ACS data, and a relatively high value for the ACS data, respectively. Thus one of the one or more parameters utilized in multifactorial analysis may be based upon the planarity of one or more vector cardiogram loops relative to a reference plane, where the loops are any of the P, QRS, or T loops, or segments thereof, as computed in the 3-D representation of the EKG data.

Referring to FIG. 7, EKG signal analysis known as Pardee analysis, named after Harold Pardee's research in the 1920's, may be utilized to generate a parameter. In essence, if the a line (98) drawn between the J point (96) and the apex of the T wave (94) shows a convex or straight ST signal (100), the patient is more likely to have a myocardial ischemia or infarction that is a patient with a concave ST signal (102) in the same location, and thus this Pardee parameter is useful in clinical diagnosis of ACS.

Referring to FIG. 8, we have created a parameter we refer to as “Gamma 2D”, which we find to be clinically valuable. Benign early repolarization (“BER”) is a condition that a particular patient will either have or not have. It is also one of the most frequent confounders of 12-lead EKG analysis that causes false positive diagnoses of ACS in clinical settings. We have found that the theta and phi (the angular coordinates of the ST vector) are very tightly clustered for a BER patient group, and very distributed for non-BER patients. Thus, we find the Gamma 2D marker, which is the position of the ST vector (106) relative to the center of the early repolarization distribution (106), to be a useful parameter. In another variation, the Gamma 2D marker may be defined as the position of the T vector (not shown) relative to the center of the early repolarization distribution. For clarity of terminology, a first cardiac condition will be used in reference to a cardiac condition that a clinician is trying to detect, while a second cardiac condition will be used in reference to a confounding condition (for example, BER, LVH and RBBB are three particular confounding second conditions that may be of interest). An objective is to eliminate the confounding problem to improve the performance of detection of the first condition. In some variations, other second conditions such as left ventricular hypertrophy (LVH) or right bundle branch block (RBBB) may be used to establish the centerpoint of the distribution. The ST vector is a vector constructed based on the orientation of the cardiac vector at points such as the J point, J point+40 milliseconds (ms), J point+60 ms, J point+80 ms, or J point+another temporal amount that shifts the cardiac vector towards the peak of the T wave (the “T point”), all such points represented on the 3-D representation of the EKG data. The T vector is the cardiac vector at the peak of the T wave. Alternatively, the cardiac vector orientation at the end of the T wave could be used to represent the T vector.

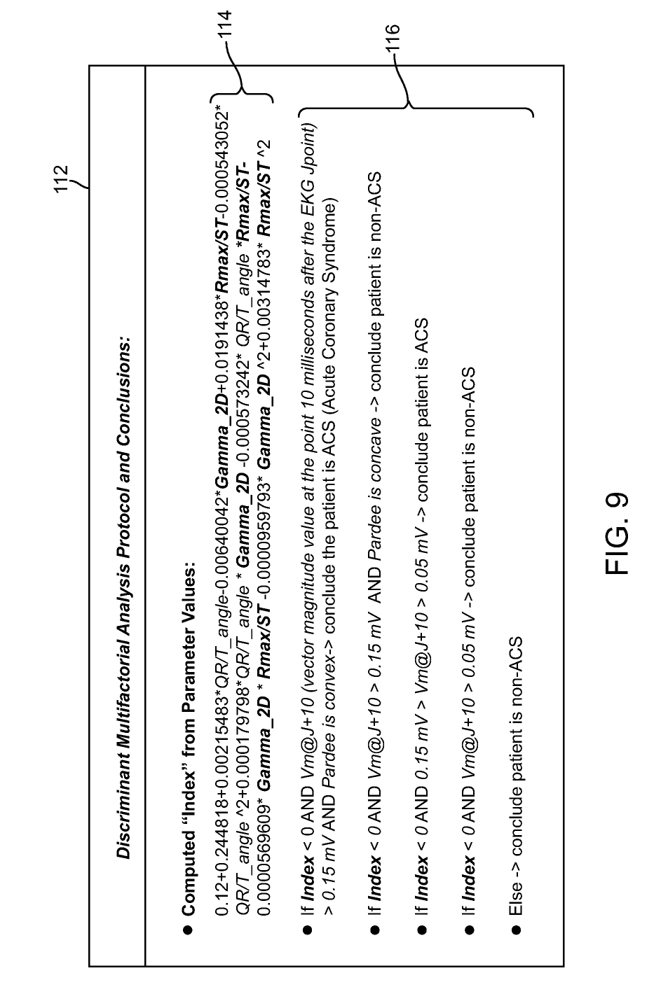

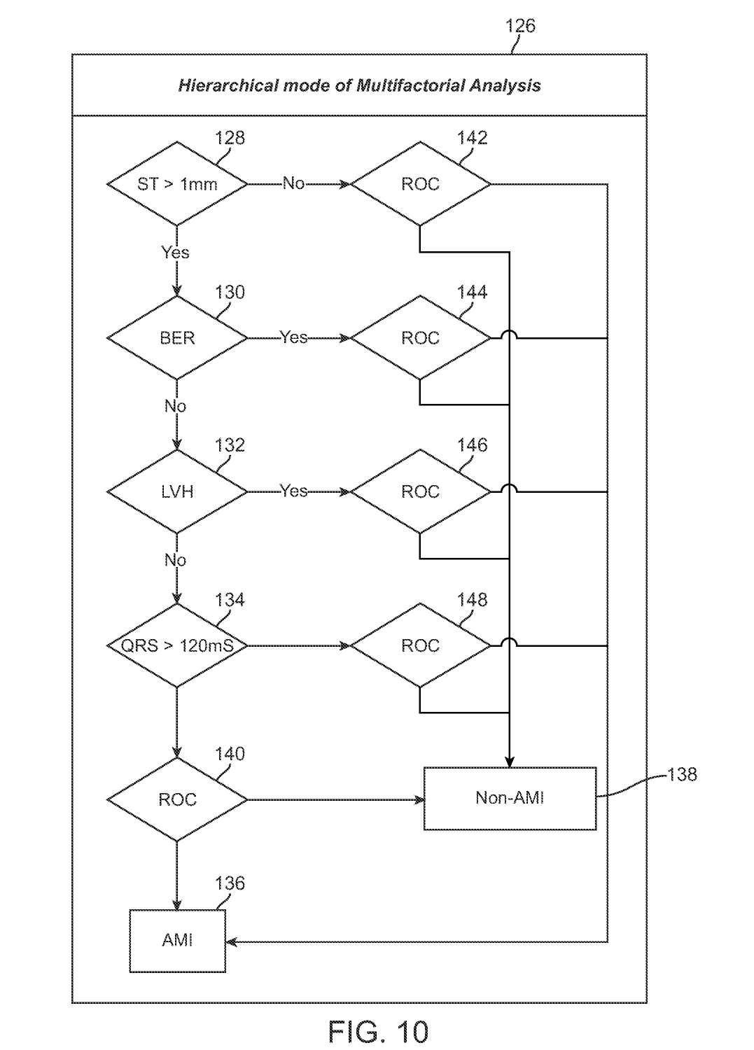

Referring to FIG. 9, one preferred embodiment of a discriminant multifactorial analysis protocol (112) is depicted, wherein calculation of a numerical “Index” based upon various parameters (QR/T angle; Gamma 2D; Rmax/ST ratio-element 114 is the equation for Index) and a series (116) of discriminant tests leads to clinical conclusions. The Index and the diagrammatic flowchart in FIG. 9 showed substantial improvement in the detection of ACS in a study performed on 460 all-corners patients that reported to an emergency department with angina. By additional clinical test (e.g. troponin tests); only 140 of these patients were confirmed to have had ACS. The algorithm represented in Figure resulted in a sensitivity of 78% and specificity of 84%. The same patients were reviewed by two expert certified, practicing cardiologists using only 12-lead EKG data. Their readings provided an averaged sensitivity of only 57% and an averaged specificity of 89%. Therefore, the algorithm improved by more than 20% the expert human reader sensitivity in detecting ACS while preserving the specificity at equivalent levels. Although preliminary, these results help to confirm that markers, parameters, protocols and algorithms according to this invention may enhance the accuracy of EKG diagnosis in emergency departments. Referring to FIG. 10, hierarchical modes of multifactorial analysis may also be utilized. For example, referring to FIG. 10, in one embodiment, if an ST elevation is above 1 mm, a receiver-operator-curve, or “decision block” (142) leads to a conclusion of non-myocardial infarction or ischemia (138); similar decision blocks (144, 146, 148, 140) are depicted for BER or not, left ventricular hypertrophy, and QRS width greater than 120 milliseconds, potentially leading to a conclusion of myocardial infarction or ischemia (136) or not (138).

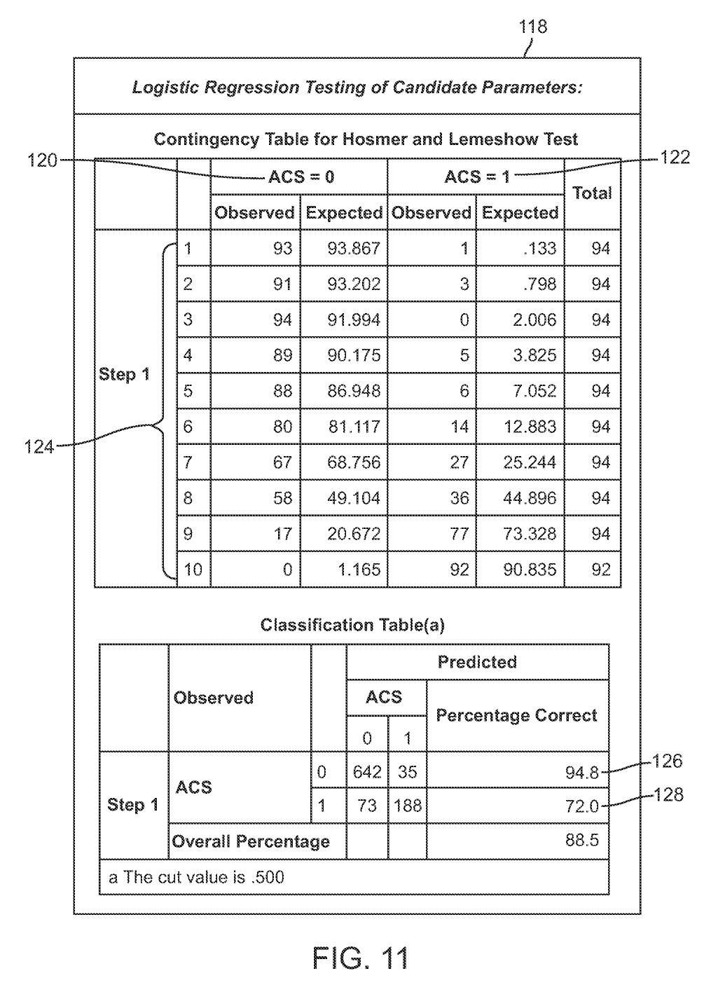

Referring to FIG. 11, a table (118) is illustrated showing how logistic regression may be utilized to test candidate parameters (124), as discussed in reference to FIG. 5. Correlation with data pertinent to an observed ACS pattern (120) and observed nonACS pattern (122) is utilized for comparisons given each of the candidate markers (124) to determine the effectiveness of each candidate marker and its contribution to overall computed specificity (126) and sensitivity (128) values.

While multiple embodiments and variations of the many aspects of the invention have been disclosed and described herein, such disclosure is provided for purposes of illustration only. For example, wherein methods and steps described above indicate certain events occurring in certain order, those of ordinary skill in the art having the benefit of this disclosure would recognize that the ordering of certain steps may be modified and that such modifications are in accordance with the variations of this invention. Additionally, certain of the steps may be performed concurrently in a parallel process when possible, as well as performed sequentially. Accordingly, embodiments are intended to exemplify alternatives, modifications, and equivalents that may fall within the scope of the claims.

1. A method for detecting a first cardiac condition, comprising:
a. providing a quantity of 12-lead EKG data from a patient;
b. constructing a 3-D representation of cardiac activity from the data;
c. computing values for one or more parameters based upon at least one of said 3-D representation of cardiac activity and said EKG data;
d. applying a multifactorial analysis protocol utilizing at least one of the values of the one or more parameters; and
e. automatically drawing one or more conclusions regarding a first cardiac condition of the patient based at least in part upon the output of the multifactorial analysis protocol.

2. The method of claim 1, further comprising acquiring the quantity of 12-lead EKG data from the patient.

3. The method of claim 1, wherein providing comprises reconstructing a 12-lead EKG recording from a reduced cardiographic vector set from the patient.

4. The method of claim 3, further comprising receiving the vector set from another device.

5. The method of claim 4, wherein the other device is selected from the group consisting of an implantable defibrillator, an implantable pacemaker, an implantable cardioverter, a portable EKG monitoring system, and a desktop EKG system.

6. The method of claim 2, wherein providing further comprises transmitting the EKG data from a plurality of electrodes to a computing system configured to construct the 3-D representation and compute values for the one or more parameters.

7. The method of claim 6, wherein transmitting the EKG data from the electrodes to the computing system occurs in real or near-real time.

8. The method of claim 6, wherein transmitting the EKG data from the electrodes to the computing system is delayed with utilization of a memory device interposed between the electrodes and computing system.

9. The method of claim 1, wherein automatically drawing one or more conclusions regarding the first cardiac condition of the patient further comprises categorizing the patient into a specific category of acute coronary syndrome relative to other patients.

10. The method of claim 1, wherein the electrodes and computing system are interfaced with a connection selected from the group consisting of a local wired connection, a local wireless connection, a remote wired connection, and a remote wireless connection.

11. The method of claim 1, wherein the multifactorial analysis protocol further utilizes one or more parameters computed directly from the 12-lead EKG data.

12. The method of claim 1, further comprising providing feedback to a healthcare provider regarding the conclusions.

13. The method of claim 1, further comprising computing one or more confidence indices regarding the one or more conclusions based at least in part upon the one or more parameter values.

14. The method of claim 1, wherein the multifactorial analysis protocol comprises quantitative analysis selected from the group consisting of discriminatory analysis, regression analysis, and hierarchical analysis.

15. The method of claim 1, wherein one of the one or more parameters is a ratio-metric parameter based upon two or more aspects of the EKG data.

16. The method of claim 15, wherein one of the one or more parameters is a ratio of Rmax relative to STshift or Tmax.

17. The method of claim 1, wherein one of the one or more parameters is based upon the planarity of one or more vector cardiogram loops relative to a reference plane.

18. The method of claim 1, wherein one of the one or more parameters is based upon the shape of one or more vector cardiogram loops relative to a reference shape, or relative to each other.

19. The method of claim 18, wherein the reference shape comprises a circle or ellipse.

20. The method of claim 1, wherein one of the one or more parameters is based upon the angular orientation of one or more vector cardiogram loops relative to a reference vector position, a reference plane, or relative to each other.

21. The method of claim 1, wherein one of the one or more parameters is based upon a Pardee sign analysis.

22. The method of claim 1, wherein one of the one or more parameters is based upon a vector position relative to a computed center of a population distribution representative of a second cardiac condition, the vector selected from the group consisting of ST vector and T vector.

23. The method of claim 22, wherein the second cardiac condition is selected from the group consisting of benign early repolarization, left ventricular hypertrophy, and right bundle branch block.

24. The method of claim 1, wherein the one of the one or more parameters of the multifactorial analysis protocol is selected based upon a factor included in the group consisting of a patient's age, gender, race, residency, citizenship, occupation, and profession.
